(19) United States
(12) Patent Application Publication
(10) Pub. No.: US 2026/0155242 A1
(43) Pub. Date: Jun. 4, 2026

(54) Computer(S) Configured to Provide and Train Intelligent Dialog Agents

(71) Applicant: GAIA AG, Hamburg (DE)

(72) Inventors: Tristan Zindler, Hamburg (DE); Bernhard Wellhöfer, Hamburg (DE); Mario Weiss, Hamburg (DE)

(21) Appl. No.: 18/966,928

(22) Filed: Dec. 3, 2024

(51) Int. Cl. G16H 40/20; G06F 40/30; G06Q 10/0639; G16H 20/70; G16H 40/67; G16H 80/00

(52) U.S. Cl. CPC G16H 40/20; G06F 40/30; G06Q 10/06398; G16H 20/70; G16H 40/67; G16H 80/00

(57) ABSTRACT

A system for training digital cognitive behavioral therapists includes a plurality of digital patients; a plurality of digital therapists; a database for storing, according to diagnosis, message and reply histories received from the digital patients and the digital therapists; a computer having access to the database; software executing on the computer for analyzing a message history for a particular patient to determine improvement or regression over time and/or over volume of communications through the patient messaging system and the therapist reply system; and software executing on the computer for ranking therapists by diagnosis and providing samples of higher ranked replies.

### TECHNICAL FIELD

The present disclosure relates to computers that are configured to provide interactive dialog experiences (“chat bots”), and more particularly, to improvements in the algorithms that chat bots use to provide such experiences.

### BACKGROUND

Presently, generative artificial intelligence systems (“GenAI”) are prevalent. Such systems use statistical guessing to produce a most likely correct reply to a prompt. They lack rigor in the algorithms that generate their replys, and they are prone to mistaken guesses called “hallucinations”. For specialized tasks that an LLM is not specifically trained on, there is not a high likelihood that a particular reply will be “correct” in a useful sense of that term.

An example of a specialized task is the training of Cognitive Behavioral Therapy (“CBT”) therapists. CBT sometimes may be referred to as “talk therapy.” In the CBT treatment modality, a patient or client converses with a trained professional to enhance the patient's functioning with any of a range of psychological disorders including depression, PTSD, etc.

Treatment sessions are traditionally held in person in a comfortable setting between therapist and patient in order to promote communication and engagement. The treatment seeks to help patients become more self-aware and recognize factors that influence their emotional well-being, and also to encourage supportive behaviors and activities so that patients can reach and maintain their own emotional balance.

It is generally known that a series of regular CBT sessions is necessary to reinforce treatment initiatives until patients themselves become aware of improvements in their emotional state.

Ubiquitous Internet connectivity and the rise of mobile computing devices have made it possible for CBT to be consumed by patients without actually visiting a therapists' office. The patient's own environment and schedule can be more easily accommodated to not only leverage the patient's own comfort, but also to expand delivery of CBT services.

Chat-bot systems are known, including systems for providing CBT, but these systems are only “intelligent” in the sense that they have the ability to answer a limited number of questions or provide a limited amount of information. Additionally, such systems have been only text-based. They either cannot accept inputs other than text, or they only provide replies in text, or both. So, the efficacy of existing chat-bot style CBT systems is limited at least because the full range of a therapists' observations and experience cannot be used for treatment.

Further, current generation chat-bots have trouble with logic and reasoning because they are fundamentally statistical guessing machines that produce the most “likely” response to a prompt. For example, chat-bots frequently have trouble counting how many “r” letters are in the word “strawberry”. The reasons for these troubles are fairly simple and also are fundamental to how chat-bots work (chat-bots represent “strawberry” as two or three tokens and count how many tokens have an “r”). When handling signals of intense emotional valence rather than spelling words, the potential for over-simplification by an unsupervised chat-bot could be counter-productive, even dangerous.

It would be desirable to provide a chat bot that could generate responses with a relatively high likelihood of being “correct” as in usable for a results-sensitive purpose. Examples of results-sensitive purposes include dialogs that provide an end-user with therapeutic or advisory results; e.g., talk therapy, legal counseling, business advisement.

### SUMMARY

According to aspects of the present disclosure, a chat bot system is provided that includes a mobile computing device; a messaging app running on the mobile computing device; a computer with access to the messaging app; a communication channel established between the mobile computing device and the computer; a message received by the computer from the mobile computing device over the communication channel; software executing in the computer for extracting at least one of audio, video and text data from the message, and for producing a summary; a database accessible by the computer for storing a history of the messages and summaries; software executing in the computer with access to the database (and, optionally, environmental factors) for generating an assessment of the message or of a conversation including the message; a database of curated replies, which may be processed to generate a numerical representation of each reply (optionally, the numerical representations may be stored with the replies); software executing in the computer for numerically representing the assessment (and/or the message) and for matching the assessment (and/or the message) to one or more replies within the database, based on distances between the numerical representations; software executing in the computer for generating a reply to the message using at least one of the message, the summary, the assessment, and/or any matching curated content; and software executing in the computer for skeptically analyzing the reply and either returning it to the reply generating software for revision or forwarding it to the communication channel for transmission to the mobile computing device; wherein, once the reply is forwarded to the communication channel it is added to the database of messages to update a conversation including the message and the reply.

According to other aspects of the present disclosure, a chat bot system is provided that includes a summarizer that is configured to receive one or more messages from a partner in at least one of audio, video, and text modalities, wherein the summarizer is further configured to produce and update a case summary based at least on the one or more messages; an inner voice that is configured to produce and update an assessment of the situation based at least on the case summary and a set of professional knowledge; and a composer that is configured to produce a reply to the partner based at least on the case summary and the assessment.

According to another aspect of the present disclosure, the chat bot system may include a supervisor that is configured to provide feedback to the composer regarding the reply, wherein the composer is further configured to update the reply in response to the feedback. For example, the supervisor may be configured to provide the feedback based at least on the set of professional knowledge.

According to another aspect of the present disclosure, the chat bot system may include a curated content injection system that is configured to receive the assessment and to provide curated content to the composer based at least on the assessment.

According to another aspect of the present disclosure, the summarizer may be further configured to produce and update the case summary based also on environmental factors.

According to another aspect of the present disclosure, the inner voice may be further configured to provide at least one motivational question to the composer based at least on the case summary, the set of professional knowledge, and the assessment.

According to another aspect of the present disclosure, the summarizer may be further configured to provide at least one gap-filling question to the composer based at least on the case summary.

According to another aspect of the present disclosure, the summarizer also may be configured to provide the at least one gap-filling question based also on the assessment.

Thus, aspects of the present disclosure can provide a professional simulator system that is available 24/7 to provide conversation partners with continuous contact. The system can be realized through a mobile text interface, for example, by texting a given number. Given the capabilities of speech-to-text and text-to-speech, as well as the ability for speaking video generation from 2-D still images and text, voice and video interfaces also are contemplated.

Such a chat bot can provide partners timely and consistent support, regardless of time or location. By using advanced agent-based systems to deliver personalized responses, the chat bot can focus on the individualized needs of partners, enhancing the accessibility and effectiveness of support.

Embodiments of a professional simulator system according to the present disclosure are not limited to a specific mode of communication. Such a chat bot can support various communication platforms, such as a proprietary web app, WhatsApp, SMS (Simple Message Service), RCS (Rich Communication Services), iMessages, Signal, FaceTime or other text, voice, and/or video modalities. Thus, a professional simulator system according to aspects of the present disclosure may allow partners to choose their preferred communication method. Speech-to-text, text-to-speech, and text-to-video technologies enable consistent and seamless interaction across different platforms, and enhance accessibility by catering to diverse user preferences and needs. The disclosed professional simulator system delivers a cohesive user experience regardless of the communication channel used.

A multi-agent approach is a key aspect of the present disclosure. In the chat bot interaction, each reply is computed not in a single step but through a complex interplay of multiple agents. These agents distribute intermediate “cognitive” steps across multiple specialized requests to generate a supportive reply. Each agent is specialized in handling specific aspects of the reply-generation task, contributing to a more accurate and efficient overall response. The system can adapt to different support scenarios by reconfiguring the agents and their interactions. By distributing tasks among multiple agents, the system enhances resilience and fault tolerance, reducing the impact of any single point of failure. Specialized agents improve the likelihood that each aspect of the support algorithm is addressed with the highest level of expertise, improving the overall accuracy and effectiveness.

Key agents include a summarizer, an inner voice, a curated content injector, a composer, and a supervisor.

The summarizer is configured to generate a diagnostic narrative from a series of messages and replies. Thus, the summarizer forms a summary of the case or conversation between the chat bot and the partner. The summarizer also forms a partner profile, a comprehensive vector of relevant characteristics across various categories or dimensions of persona, demographics, goals, and limitations. Additionally, the summarizer detects and/or predicts missing information and generates anamnesis (guided recall) questions that can be fed to the composer. Overall, the summarizer provides a long-term memory representation of the chat bot's interaction with the partner. As part of the long-term memory representation, the summarizer compresses the information from the messages and replies into a compact vector that can be fed to the composer. The compressed information enables maintenance of continuity in the conversation by keeping track of the partner's history, attributes, progress. The summarizer's representation of the interaction also enables provision of insight into the interaction. The summarizer operates in parallel to the other agents, so that its algorithm does not drive latency in the conversation.

The inner voice is configured to represent the cognitive process of an expert interlocutor. As such, the inner voice combines all available partner information (including the summarizer's representation of such information) with relevant professional knowledge to provide an expert assessment of the interaction and the partner's situation. Based on the expert assessment, the inner voice proposes relevant questions and/or suggestions that could be posed to the partner. The inner voice thereby plans a further course of action in the conversation. The inner voice operates independently of the summarizer, composer, and supervisor, working in parallel rather than sequentially. Once the inner voice formulates a new assessment, the assessment is stored in a history of the interaction for access and use by the composer. The inner voice, by operating in parallel to the other agents to plan the course of the conversation, enhances response speed from the partner's perspective by preparing assessments ahead of time. Unlike a human conversation, the chat bot is fully capable of both receiving a message and planning a response in parallel. Thus, the inner voice enables enhanced or superior active listening.

The curated content injector (“CCI”) is an agent that responds to the expert assessment produced by the inner voice. The CCI provides pre-curated content elements such as: relaxation audios; in-depth motivational or information-seeking questions; conversational interventions; educational content; and/or instructions for responding to crises (e.g., in a business context, cash receipts less than cash expenses; in a psychotherapeutic context, suicidal ideation). The curated content can include, e.g., audio, image, and text elements in any combination; videos and interactive elements. The CCI is intended to ensure that the partner receives pre-authored, well-targeted content exactly as intended by the authors. The CCI matches content to the partner's situation based on a numerical matching (e.g., cosine distance) between a vector embedding of the inner voice's expert assessment and vector embeddings of the curated content. Thus, the CCI provides a dynamic, automatic selection of conversational interventions and content, unprecedented in its capabilities. For example, the CCI may select the top five content elements that best fit (cosine distance match) the embedding of the current assessment of the partner's situation. The CCI then may provide these selected contents to the composer for potential inclusion in the response. In some embodiments, the composer may be obliged to include the curated content. In other embodiments, it may be optional for the composer to include the curated content.

The composer formulates drafts for a reply to the partner's message, based on all available information about the partner including the message itself, the inner voice's assessment and the summarizer's representation of the interaction with the partner. Thus, the composer utilizes information partially prepared by other agents. The composer tailors each reply to the specific needs and context of the partner. The composer maintains consistency in the conversation by harmonizing data from the other agents.

The composer does not send replies directly to the partner; instead, the supervisor reviews every reply and occasionally provides feedback to the composer. The supervisor generates feedback based on a set of relevant professional knowledge, which may be the same professional knowledge that is used by the inner voice. Checking replies against professional knowledge can help to make replies appropriate within the context of the conversation. Thus, the supervisor can protect the professional simulator system against prompt injections and various malicious user requests. The supervisor is responsible for system boundary maintenance by ensuring that the overall system remains within the defined scope of the chat bot's assigned purpose. The supervisor enhances the quality and safety of the chat bot's replies, maintains consistent standards of expertise, and safeguards against potential misuse or harmful responses.

In response to feedback from the supervisor, the composer may produce revised draft replies.

There is also a need for a client simulator system employing many of concepts described herein. Such a client simulator system might operate using a large language model as a base to mimic human behavior.

Possible use cases for a client simulator system include serving as a test system for a professional simulator system according to the present teachings, as well as a system for generating training data for said system, and as a coaching system for human therapists.

A client simulator system according to the present teachings might be programmed to reflect different “characters.” For example, and without limitation, one such character could mimic a human patient who was unstable, impulsive, insecure, histrionic, anxious, and avoidant. Another character could mimic a human patient who was depressed, lonely, suffering from chronic pain, and resistant to change. Other emotions or characteristics of human patients are readily envisioned by the present teachings, including

A client simulator system is provided that includes: a character trait database having a plurality of human character traits; a message database having a plurality of patient memories; a computer with access to the character trait database and the message database, said computer having a communication link between said computer and a therapist; software executing on the computer for retrieving a patient memory and a set of character traits for a test patient and formulating an inner voice of the test patient based on the patient memory and the character traits; software executing on the computer for combining a new message with the inner voice to compose a reply of the test patient; software executing on the computer to update the patient memory based on the new message and the reply; and transmitting the revised message to the therapist using the communication link.

Key agents include a memory, a character traits database, an inner voice, and a composer.

The memory is configured to generate a narrative from a series of messages and replies. Thus, the memory forms a summary of the case or conversation between the patient and the trainee. Overall, the memory provides a long-term memory representation of the patient's interaction with the trainee. The memory of the digital patient is generated by an LLM agent by summarizing the conversation with the trainee/therapist from the patient's perspective. The summarized or compressed information enables maintenance of continuity in the conversation by keeping track of the trainee's history, attributes, progress. The memory's representation of the interaction also enables provision of insight into the interaction. The memory operates in parallel to the other agents, so that its algorithm does not drive latency in the conversation.

The inner voice is a representation of the patient's inner monologue, which expresses the struggle, thoughts and feelings the patient has during therapy. Thus, the inner voice utilizes information from the memory and from the character traits database. The inner voice maintains consistency in the conversation.

The composer is configured to represent the cognitive process of a professional's client, e.g., a behavioral treatment therapy client. As such, the composer generates the test patient's reply to a new message from the trainee by combining the new message with the inner voice representing the current state of mind of the patient. However, the memory and inner voice of the patient are updated asynchronously and thus do not delay the generation of the reply. Once the composer formulates a reply, the reply is stored in the patient's memory and is transmitted to the trainee via the communication link. The composer, by operating in parallel to the other agents to plan the course of the conversation, enhances reply speed from the trainee's perspective.

It is envisioned that a professional simulator system and a client simulator system according to the present teachings may be combined such that a simulated therapy conversation can take place. Such a system will be referred to as a simulated therapy conversation system.

Many purposes for a simulated therapy conversation system are envisioned, including without limitation (1) as a means to test both short- and long-term behavior of either the professional simulator system or the client simulator system, (2) as a means to evaluate the therapeutic performance of the professional simulator system, and (3) as a means to generate synthetic training data for either the professional simulator system or for the client simulator system.

A simulated therapy conversation system according to the present teachings may include one or more of the following: a connector, a time machine, a diaglog, and a diagnos. The purpose of the connector is to seamlessly connect the professional simulator system and the client simulator system. The purpose of the time machine is to simulate progress over time. The purpose of the diaglog is to generate a realistic dialog between a patient and a therapist. The purpose of the diagnos is to evaluate local and global performance metrics of the selected therapy.

As mentioned, one purpose for using a simulated therapy conversation system is to evaluate the therapeutic performance of the professional simulator system. In particular, a goal of this use is to measure the relative performance of message and therapy progress.

Other metrics may be considered when reviewing performance of a simulated therapy conversation system, including local quality metrics such as therapist metrics (e.g., does the composer's message fulfill effective therapeutic practices?) and measures concerning the patient's emotions (e.g., how does the composer's message impact the patient's emotions?). Other metrics may take into account the full history of a simulated conversation between the professional simulator system and the client simulator system, including goal completion (i.e., what percentage of the therapy goals have been satisfied?), the patient's needs (i.e., are the patient's basic needs met?), resource activation (i.e., are the patient's resources utilized during therapy), and the overall therapeutic relationship (i.e., does the therapist develop an effective relationship with the patient?).

When measuring the local quality of a therapeutic treatment, a system according to the present teachings may define key attributes of effective therapeutic praxis for formulating responses. Attributes may be one or more of the following: engaging, helpful, language, emphatic, actionable, relevant, accurate, appropriate, accepting, clear, and empowering. The attributes can individually be given grades of agreement or disagreement. It is contemplated that said attributes can be defined on a nine-point scale, though other configurations are within the scope of the present teachings.

Measuring the patient's emotions is also contemplated as assessing a variety of attributes using a scaled system. Attributes for patients may include, but are not limited to, the patient's emotional spectrum, the patient's development, and the patient's development. A full spectrum of emotions based on semantic space theory is contemplated. Metrics of a patient's emotions are purely text-driven on a scale which may range from 0 to 10 or another relevant scale.

To measure the relative goal completion across the full history of a simulated conversation between a professional simulator system and a client simulator system, goals are preferably initially defined. Measured goals may be short-, medium-, or long-term, and progress indicators are contemplated. To measure completion, a system according to the present teachings may be capable of estimating the fulfilled percentage of each goal.

A system according to the present teachings may be capable of measuring a hypothetical patient's needs, the purpose of which is to assess how well a patient's needs are being met/have been met during the course of their therapy. One such means, though others may be applicable, for measuring how well a patient's needs have been met is to categorize the patient's answer(s) into 9 grades of agreement or disagreement and provide a score ranging from −1 to 1. For example, a patient's answers may be assessed on needs including physiological, safety, love and belonging, esteem, and self-actualization. Autonomy, relatedness, and competence are also contemplated as measurable.

To measure the resource activation of a system according to the present teachings, one embodiment may categorize a patient's (or a client simulator system's) answer into multiple (e.g., 9) grades of agreement or disagreement and provide a score ranging from −1 to 1, though other ranges are appropriate. Resource categories may include, but are not limited to, completeness, diversity, sufficiency for therapy, activation frequency, integration into therapy strategy, impact on the patient's progress, and activation of identified resources.

Measuring the therapeutic relationship of a simulated therapy conversation system is meant to provide a comprehensive analysis for evaluating the quality of the therapeutic relationship. It is envisioned that the patient's (or the client simulator system's) answers can be categorized into multiple (e.g., 9) grades of agreement or disagreement, with each category given a score from −1 to 1, though other ranges are appropriate. Categories may include, but are not limited to, effectiveness, goal alignment, emotional attunement, communication clarity, cultural sensitivity, personalization, progress reflection, supportiveness, crisis management, therapeutic alliance, empathy, validation, approach alignment, and flexibility.

It is contemplated that a system incorporating a professional simulator system, a client simulator system, and a simulated therapy conversation system according to the present teachings may be used as a means for generating valuable training data for the professional simulator system and/or other systems. A professional simulator system according to the present teachings functions as a digital therapist. A client simulator system according to the present teachings functions as a client simulator. A simulated therapy conversation system functions as a system connecting the professional simulator system and the client simulator system. A system incorporating a professional simulator system, a client simulator system, and a simulated therapy conversation system according to the present teachings will be referred to as a digital therapy leaning loop.

A digital therapy leaning loop is envisioned as a continuous feedback system, wherein the simulated therapy conversation system captures and logs interactions between the professional simulator system and the client simulator system. These interactions are stored in a database or other appropriate source and form a collection of data. This collection of data can then be evaluated using the metrics described herein. Based on the evaluation results, any individual system (i.e., chat bot, client simulator, simulated therapy conversation) can be adjusted. As more interactions, and therefore more evaluations, become available, the digital therapy learning loop provides a means for continuous improvement of any individual system.

Other features and aspects of the present teachings will become apparent from the following detailed description, taken in conjunction with the accompanying drawings, which illustrate by way of example the features in accordance with embodiments of the present teachings. The summary is not intended to limit the scope of the present teachings.

### BRIEF DESCRIPTION OF THE DRAWINGS

The present teachings are described more fully hereafter with reference to the accompanying drawings, which depict example embodiments. The following description illustrates the present teachings by way of example, not by way of limitation of the principles of the present teachings.

FIG. 1 depicts an overall interaction 100 of a professional simulator system 101 with a client simulator system 601, consistent with selected aspects of the disclosure.

FIG. 2 depicts details of the professional simulator system 101.

FIG. 3 depicts inputs to a prompt 300 for an inner voice 104 of the professional simulator system 101.

FIG. 4 depicts inputs to a prompt 400 for a composer 108 of the professional simulator system 101.

FIG. 5 depicts inputs to a prompt 500 for a supervisor 110 of the professional simulator system 101.

FIG. 6 depicts an overall interaction 600 of a client simulator system 601 with a professional simulator system 101, consistent with selected aspects of the disclosure.

FIG. 7 depicts layers of the client simulator 601 memory 612.

FIG. 8 depicts inputs to an inner voice 602 of the client simulator 601.

FIG. 9 depicts inputs to a composer 604 of the client simulator 601.

FIG. 10 depicts a system 1000 for evaluating a professional simulator system 101 that interacts with a client simulator system 601, consistent with selected aspects of the disclosure.

It should be understood that throughout the drawings corresponding reference numerals indicate like or corresponding parts and features.

### DETAILED DESCRIPTION

For purposes of explanation and not limitation, specific details are set forth such as particular structures, architectures, interfaces, techniques, etc. in order to provide a thorough understanding. In other instances, detailed descriptions of well-known devices and/or methods are omitted so as not to obscure the description with unnecessary detail.

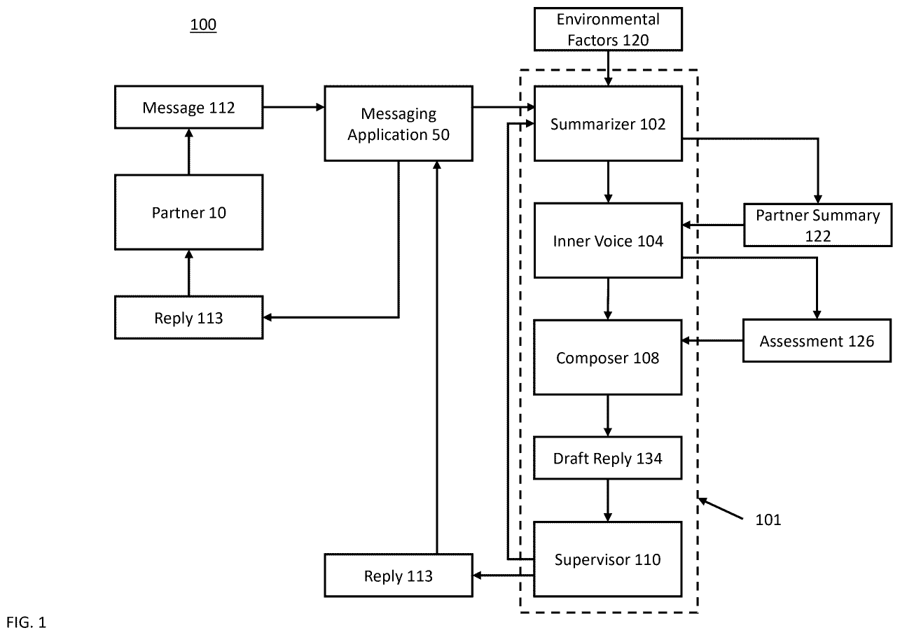

FIG. 1 depicts an overall interaction 100 of a professional simulator system 101 with a partner 10, consistent with selected aspects of the disclosure. The professional simulator system 101 repeatedly receives a message 112 from the partner 10 (e.g., a therapy patient), via a messaging app 50, and delivers a reply 113 to the partner 10. A supervisor 110 may intermediate the reply 113 and provide feedback to the professional simulator system 101. The professional simulator system 101 produces the reply 113 based on one or more of the message 112, a partner (patient) summary 122, environmental factors 120, and/or an expert (therapeutic) assessment 126.

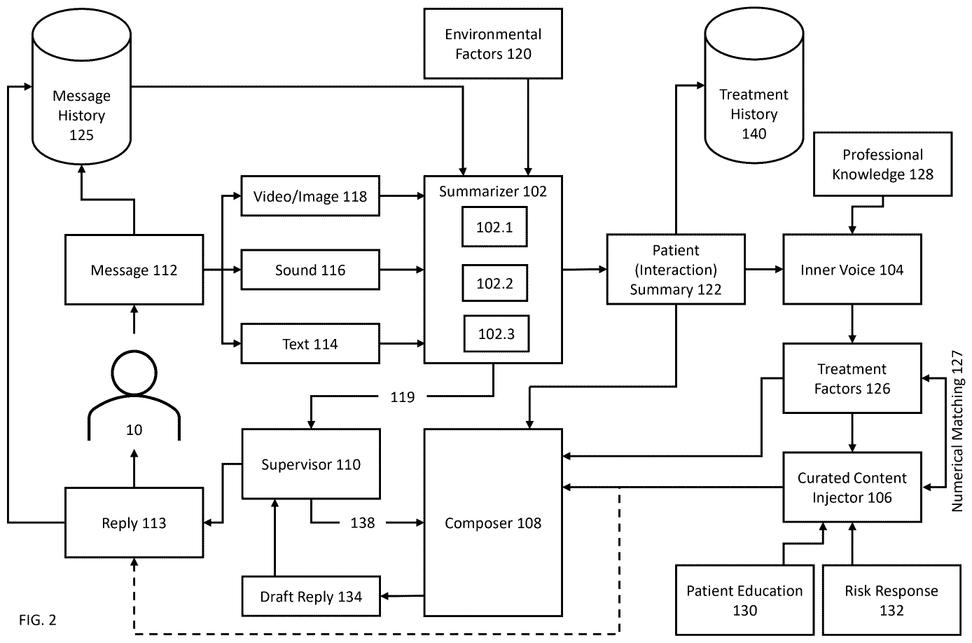

FIG. 2 depicts details of the professional simulator system 101. The professional simulator system 101 includes a summarizer 102, an inner voice 104, a curated content injector 106, a composer 108, and a supervisor 110.

In operation of the professional simulator system 101, the summarizer 102 receives the message 112 from the partner 10. The message 112 includes one or more of text 114, sound 116, and/or video/image data 118. The summarizer encodes the audio and/or video data as alt text and compiles the alt text with the message text 114 to form a full text 119. The summarizer sends the complete text 119 to the supervisor 110. The summarizer includes another encoder neural network 102.1 that is configured to compile the message 112 (optionally, in combination with sensed environmental factors 120) with one or more previous messages to produce an interaction (therapy) summary 122, which is a long-term memory representation of the interaction or conversation that the professional simulator system 101 has with the partner 10. The summarizer also includes a generative neural network 102.2 that is configured to produce a partner (patient) profile 123 based on the interaction summary 122 using weights that are encoded with professional (therapeutic) knowledge 128. The summarizer 102 also may include a generative neural network 102.3 that is configured to identify gaps or missing information in the partner summary 122 and may be further configured to generate information-seeking or anamnesis questions 124 based on the partner summary 122. The summarizer 102 may be implemented, for example, as an encoder network. The summarizer 102 also may be implemented as a portion of a long short term memory (LSTM) neural network. The summarizer 102 stores the partner summary 122 in a message history database 125 and also feeds the partner summary 122 to the inner voice 104.

The inner voice 104 is configured to generate an assessment of treatment factors 126 including the partner and the interaction with the partner, based at least on the partner summary 122 and a set of professional (therapeutic) knowledge 128. The inner voice 104 may be configured, for example, as an encoder or as a transformer network that takes at least the partner summary 122 as a prompt. The set of professional knowledge 128 may be input to the inner voice 104 as a complex (many token, e.g., thousands of tokens) prompt, and/or may be encoded in the weights of the inner voice 104 in case the inner voice 104 is implemented as a large language model (LLM) or other type of neural network. The assessment 126 may be in the form of a multi-dimensional vector that diagnoses or describes the partner and the interaction across dimensions such as persona, demographics, goals, and limitations. The inner voice 104 feeds the assessment 126 to the composer 108, and also feeds the assessment 126 to the curated content injector 106.

The curated content injector 106 may match the assessment 126 to one or more items of curated content such as partner education 130 and/or risk response information 132, in order to identify any curated content that should be imparted to the composer 108. For example, the curated content injector 106 may vectorize the assessment 126 in a semantic space and then perform vector matching (e.g., cosine distance) between the vectorized assessment and respective semantic space vectors of the curated content.

The composer 108 is configured to receive at least the message 112, the partner summary 122, and the assessment 126, as well as (optionally) curated content 130, 132. The composer 108 may be implemented as a generative adversarial neural network (“GAN”) (e.g., using transformer architecture) that takes a compilation of the message 112, the partner summary 122, and the assessment 126 as a prompt, and may take the curated content 130, 132 either as an overriding prompt or as an addition to the prompt including the other content. The composer weights may be trained on a set of situational data, questions, and suggestions. The composer 108 is configured to deliver one or more draft replies 134 to the supervisor 110.

The supervisor 110 is configured to receive the draft replies 134 from the composer 108. The supervisor 110 may be implemented as a GAN that takes only the set of professional knowledge 128 and the current message 112 as inputs, produces a set of model replies, and uses a vector distance algorithm that compares each draft reply to each of the set of model replies. In case the supervisor 110 finds no close match, then the supervisor 110 may provide feedback 138 to the composer 108, thus prompting a revised set of draft replies.

At each iteration of message 112 and reply 113, the professional simulator system 101 stores these communications in the message history 125. The professional simulator system 101 also stores a compilation of patient summaries 122 in a treatment history 140.

Prototypes of the professional simulator system, the client simulator system, and the training system operate on multiple instances of GPT-4 by OpenAI. Open-source models such as LLAMA 3 are equally suitable. The chat bot system may be self-hosted. Using multiple instances of large language models (LLMs) that take separate customized prompts and/or are trained on custom data enables the professional simulator system 101 to produce high-quality responses. LLMs can provide powerful capabilities for processing and generating human-like text. Moving to open-source models may enhance scalability and provide greater control over the system. For example, using a self-hosted open-source model may allow for customization and fine-tuning to meet specific support needs. Additionally, self-hosting ensures higher security and better privacy for user data. As an alternative or supplement to fine-tuning with data, embodiments of the chat bot system may utilize advanced prompt engineering (for example, based on an database of curated prompts) for effective responses.

In various applications, certain components of the professional simulator system 101 may serve distinct roles. For example, if the professional simulator system 101 is implemented in a psychotherapeutic role, then the partner summary 122 may be better described as a patient summary 122, while the assessment 126 may be better described as treatment factors 126. In such an application, the curated content may be better described as patient education 130 and risk response 132.

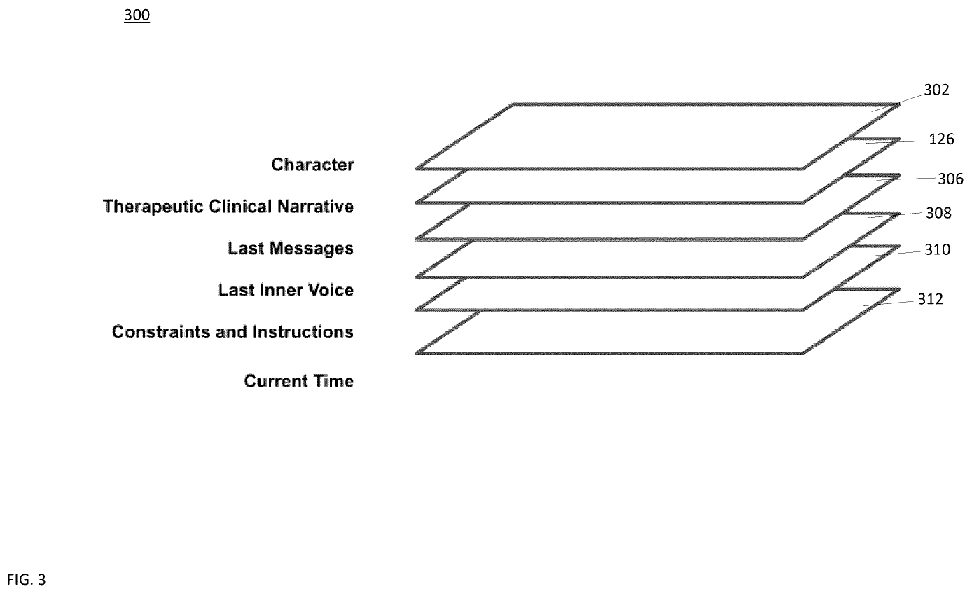

FIG. 3 depicts inputs to a prompt 300 for an inner voice 104 of the professional simulator system 101. The prompt 300 incorporates a therapeutic character 302, the therapeutic clinical narrative or assessment 126, a compilation of the last messages 306 (e.g., the six most recent messages), an echo of the last inner voice output 308, constraints and instructions 310, and a current time 312.

Options for the therapeutic character 302 include age, gender, race, education, and other aspects of a notional therapist's identity that are compiled into a framing portion of the prompt 300.

The therapeutic assessment 126 is an expert encoding or assessment of the message history as discussed above.

One purpose of the echo 308 is to maintain a continuity of context across multiple message and reply sequences.

The constraints and instructions 310 may include, for example, a constraint to acknowledge but not affirm negative messaging; a constraint to redirect attacks on the therapist/chat bot; a constraint to ignore attacks on the therapist/chat bot; an instruction to focus or perseverate on a given issue of concern to the partner/patient; an instruction to elicit additional detail from vague statements; etc.

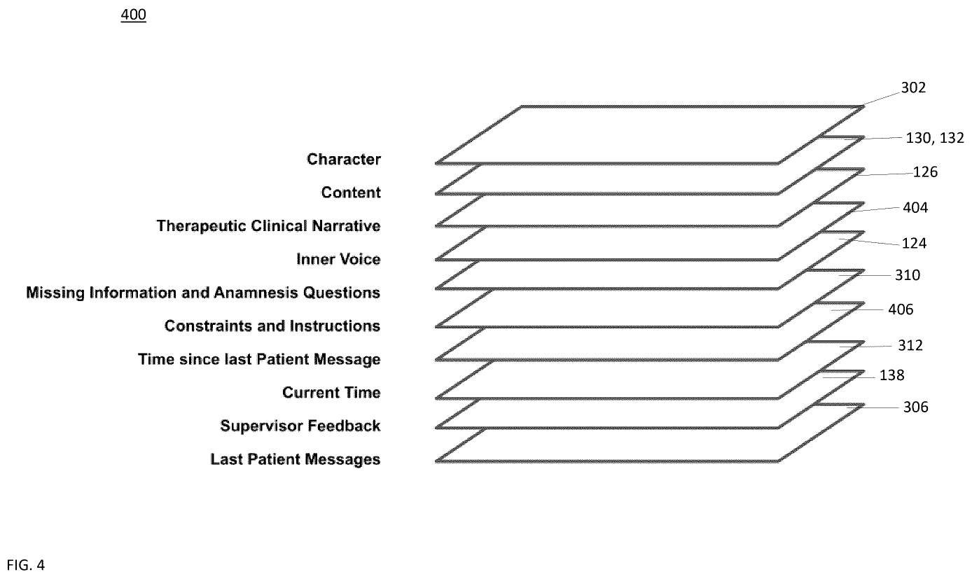

FIG. 4 depicts inputs to a prompt 400 for a composer 108 of the professional simulator system 101. The prompt 400 includes the therapeutic character 302, curated content 130, 132, therapeutic clinical narrative or assessment 126, inner voice output 404, missing information and anamnesis questions 124, constraints and instructions 310, time since last patient message 406, current time 312, supervisor feedback 138, and last patient messages 306.

As mentioned, the summarizer produces the anamnesis questions 124. The inner voice output 404 is produced by the inner voice 104 in response to the prompt 300.

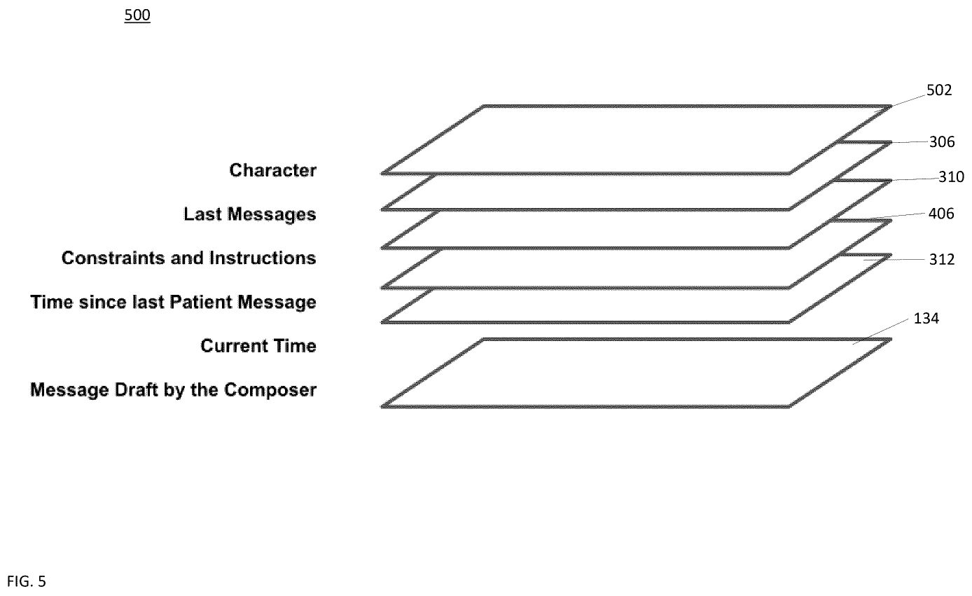

FIG. 5 depicts inputs to a prompt 500 for a supervisor 110 of the professional simulator system 101. The prompt 500 includes supervisor character 502, last messages 306, constraints and instructions 310, time since last patient message 406, current time 312, and draft reply or replies 134.

The supervisor character 502 is distinct from the therapeutic character 302 in at least one dimension of age, gender, race, education, or other identity factors. Advantageously, this gives the effect of multiple perspectives on the task at hand.

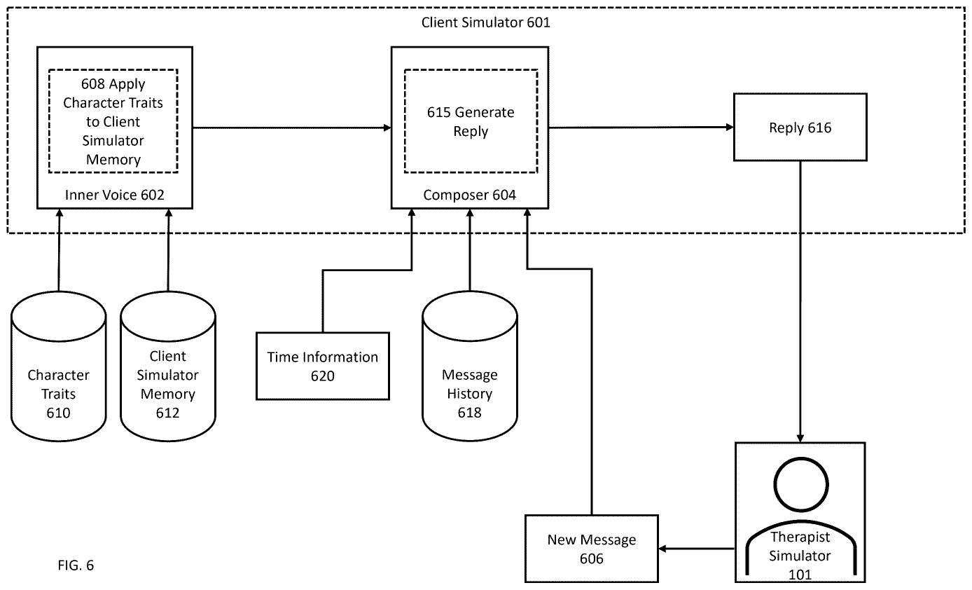

FIG. 6 depicts an overall interaction 600 of a client simulator 601 with a therapist simulator 101, consistent with selected aspects of the disclosure. The client simulator 601 includes an inner voice 602 and a composer 604. The client simulator receives a new message 606 from the therapist simulator 101 and produces a reply 616.

First the client simulator 601 formulates the inner voice 602 by applying 608 character traits 610 to a patient memory 612. For example, the inner voice 602 uses the character traits 610 as a context portion of a complex LLM prompt and uses the patient memory 612 as a situation portion of the complex LLM prompt where the task is to produce the inner voice 602: (“You are a person who [character traits=context] and you have had the following conversations: [=situation]. Tell me what you are thinking [=task].”). For example, the set of character traits 610 may be in the form of a complex (many token, e.g., thousands of tokens) prompt. Alternatively, the character traits 610 may be encoded in the weights of a neural network in the patient 601.

Next, the composer 604 generates 614 the reply 616, based at least on the inner voice 602, the new message 606, a history of messages 618, and time information 620. The composer 604 applies the inner voice 602 to the new message 606 in order to generate the reply 616. For example, the composer 604 may use the inner voice 602 as a context portion of a complex LLM prompt and may use the new message 606 as a situation portion o the complex LLM prompt where the task is to produce the reply 616: (“You are thinking [inner voice 602=context] and the therapist says [new message 606=situation]. What do you say next? [=task]”).

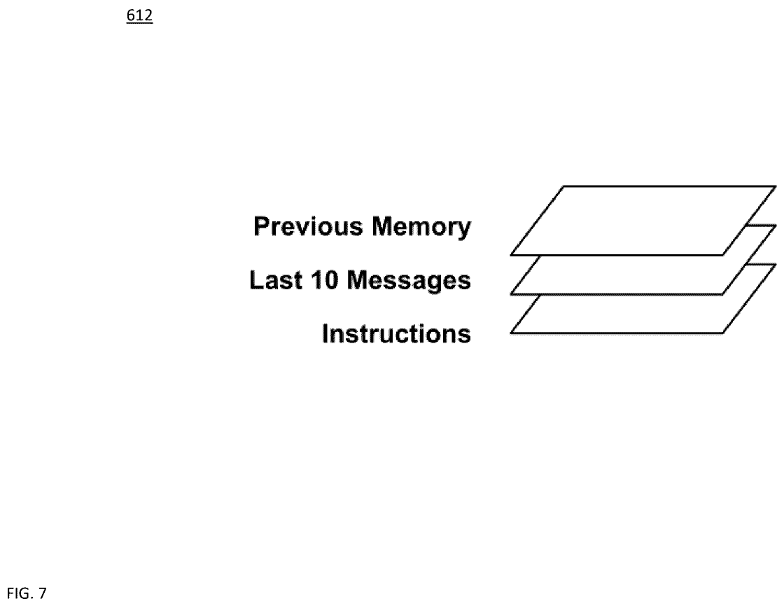

At each iteration of new message 606 and reply 616, the client simulator 601 stores these communications in the memory 612. FIG. 7 depicts how the client simulator 601 stores communications in the memory 612. The layers of the memory 612 include previous memory (a compressed or encoded version of the patient memory 612 plus character traits 610), the last ten or however many messages (ten messages being an example buffer window for convenience of understanding), and instructions (e.g., the current character traits 610). The memory 612 is used as part of a prompt to generate the inner voice 602. Purposes of the memory include compressing the therapy conversation, mitigating “whispers”, extracting details from the conversation, tracking trainee experience and patient progress, goal and task tracking, and identification of information gaps.

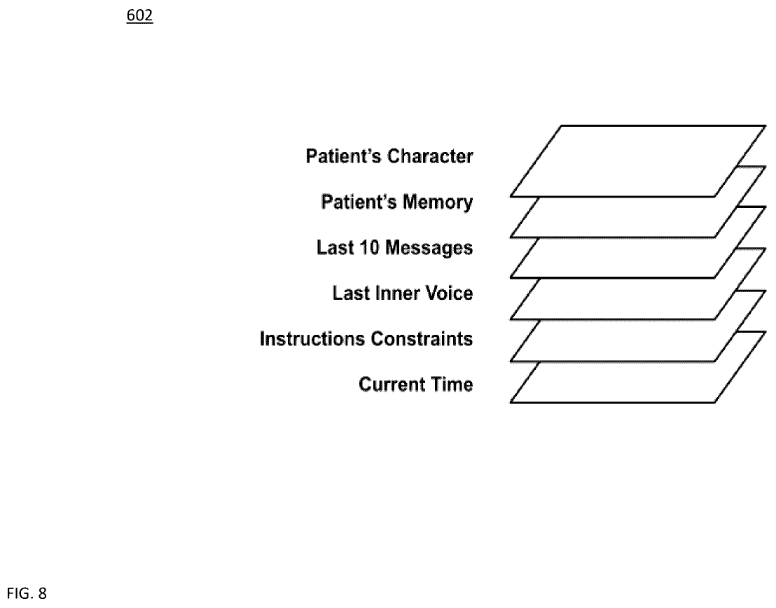

FIG. 8 depicts inputs to a prompt 800 for the inner voice 602 of the patient 601. Purposes of the inner voice 602 include creating a patient's inner monologue; emulating cognition of the patient; expressing struggle, thoughts, and feelings; maintaining character consistency across messages 616; maintaining continuity of the conversation; and keeping aware of time. The prompts include the character traits 610, the memory 612, the last ten messages, the last composer, instructions and constraints, and a current time. Each iteration of the composer updates the previous composer.

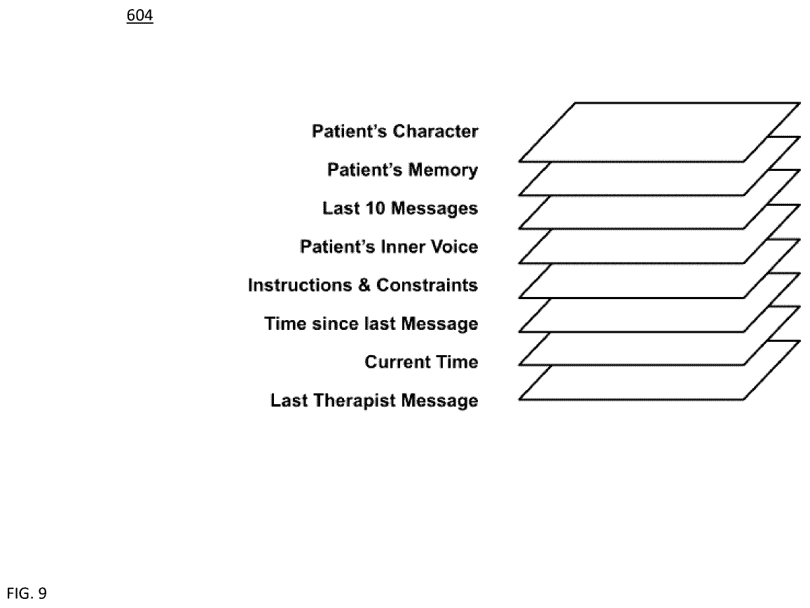

FIG. 9 depicts inputs to a prompt (00 for the composer 604 of the patient 601. The purpose of the composer is to produce a “realistic” reply 616 as a patient's answer to the therapist simulator's new message 606. Instructions and constraints for the composer include language and style; structure and content; contextual awareness; interaction dynamics; and behavioral realism. Accordingly, the composer 604 incorporates the character traits 610, the memory 612, the last ten messages, the inner voice 602, the aforementioned instructions and constraints, a time since the last message, a current time, and the new message 606.

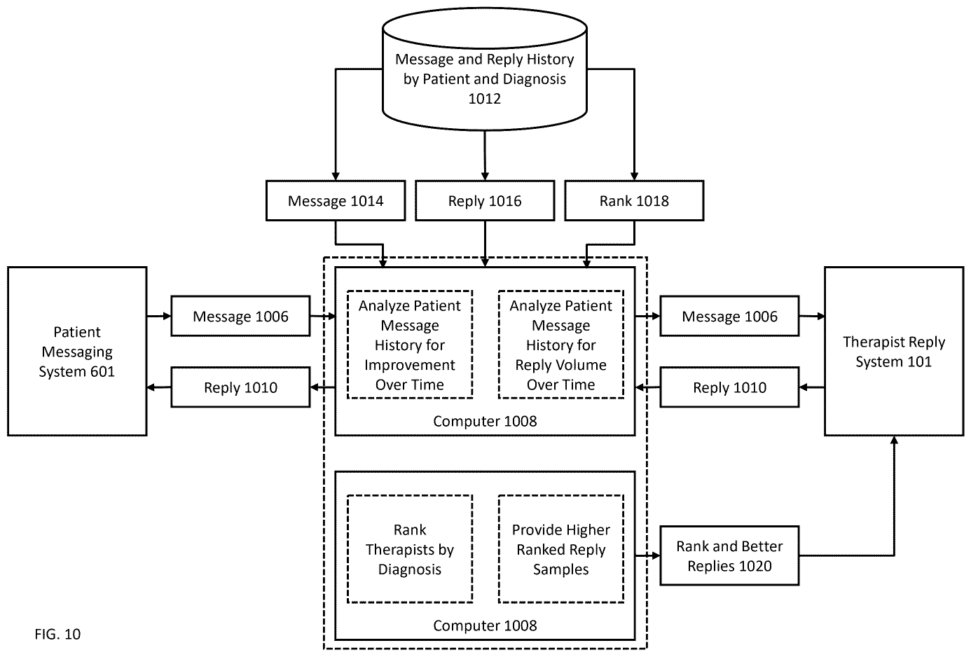

FIG. 10 depicts a system 1000 for evaluating a professional simulator system 101 that interacts with a client simulator system 601, consistent with selected aspects of the disclosure.

The system 1000 interacts between a patient (e.g., a patient messaging system) 601 and a therapist (e.g., a therapist reply system) 101. The system 1000 receives a message 1006 from the patient messaging system 601, processes the message 1006 in a computer 1008, and transmits the message 1006 to the therapist reply system 101. The system 1000 also receives a reply 1010 from the therapist reply system 101, processes the reply 1010 in the computer 1008, and transmits the reply 1010 to the patient messaging system 601.

The computer 1008 accesses a database 1012 that stores message and reply history by patient and diagnosis. From the database 1012, the computer 1008 retrieves messages 1014, replies 1016, and rankings 1018. The computer 1008 applies the information retrieved from the database 1012 to analyze the patient message history for improvement over time and to analyze the patient message history for reply volume over time.

For example, the computer 1008 may analyze the patient message history to measure relative performance of messages and therapy progress. The computer 1008 may analyze the patient message history to produce an objective measure for impact of incremental changes made to the therapist reply system and/or patient messaging system. The computer 1008 may analyze the patient message history to assess a patient's state, development and progress in therapy.

The computer 1008 may analyze the patient message history to produce various metrics, including for example composer quality (single message) or a variety of therapist metrics. Therapist metrics may be on a scale of agreement (e.g., nine bins from negative to positive) or on a numeric score between 0 and 10 or between 0 and 100, and may include answers to questions such as:
To what extent does the reply fulfill good therapeutic practice?
To what extent does the reply impact patient's emotions?
To what extent is the patient progressing in therapy? What is percentage of therapy goals completed?
Are the patient's needs met?
Are patient's resources utilized during therapy?
To what extent do the therapist and patient develop a functional relationship?

Regarding the measure “To what extent does the reply fulfill good therapeutic practice”, the computer 1008 may assess each reply against key attributes of good therapeutic praxis. For example, the computer 1008 may use the following eleven mostly orthogonal attributes: engaging, helpful, language, empathic, actionable, relevant, accurate, appropriate, accepting, clear, empowering. These may be assessed based on natural language processing (NLP) of each reply, e.g., by latent semantic analysis (LSA), to categorize each reply into one of a plurality of grades (e.g., nine grades) of agreement/disagreement with each of the attributes.

Regarding the measure “To what extent does the reply impact patient's emotions?”, patient emotions, in a spectrum of 52 types, are detected purely text-driven using an LLM and scored on a scale from 1 to 10 based on single messages of a patient. Emotional shifts, activation and long-term change during therapy are performance measures with respect to a reference for the digital therapist. The computer 1008 may employ natural language processing, such as LSA, to assess the patient's emotional spectrum, development, and activation. Semantic space theory (SST) may be adapted to measure a full spectrum of emotions, e.g., 52 different emotions. The 52 emotions are: [‘admiration’, ‘adoration’, ‘aesthetic_appreciation’, ‘amusement’, ‘anger’, ‘annoyance’, ‘anxiety’, ‘awe’, ‘awkwardness’, ‘boredom’, ‘calmness’, ‘concentration’, ‘confusion’, ‘contemplation’, ‘contempt’, ‘contentment’, ‘craving’, ‘desire’, ‘determination’, ‘disappointment’, ‘disapproval’, ‘disgust’, ‘distress’, ‘doubt’, ‘ecstasy’, ‘embarrassment’, ‘empathic_pain’, ‘enthusiasm’, ‘entrancement’, ‘envy’, ‘excitement’, ‘fear’, ‘gratitude’, ‘guilt’, ‘horror’, ‘interest’, ‘joy’, ‘love’, ‘nostalgia’, ‘pain’, ‘pride’, ‘realization’, ‘relief’, ‘romance’, ‘sadness’, ‘sarcasm’, ‘satisfaction’, ‘shame’, ‘surprise_negative’, ‘surprise_positive’, ‘sympathy’, ‘tiredness’, ‘triumph’] which is based on the Semantic Space Theory as published in https://www.cell.com/trends/cognitive-sciences/abstract/S1364-6613 (20) 30276-X.

SST measurements may be purely text-driven on a scale [0,10] with chain-of-thoughts. Emotions measured by SST may be compared to expert reference, emotional activation and shifts. The concept of shifts is that by tracking the strength of emotions over time certain emotions might be activated during therapy or the person's emotional spectrum might change significantly over time (emotion shift), e.g. from sadness to acceptance during grieving. Both are indicators for therapeutic progress.

Regarding the measures “To what extent is the patient progressing in therapy? What is percentage of therapy goals completed?” The computer 1008 may implement a complex LLM goal generator to define N short/mid/long-term SMART (Specific, Measurable, Achievable, Relevant, and Time-Bound) goals obtained by NLP of the therapist replies and patient messages. Based on the patient character description an analysis of the patient's underlying psychological issues is performed and a set of short-/mid-/long-term therapy goals and progress indicators are formulated using SMART principles. For each possible goal the progress is evaluated based on incremental updates of the therapy message history and the completion level on a scale from 0 to 100 is provided together with its supporting evidence. An averaged completion percentage of all therapy goals is used as an estimate for therapy progress. For a progress indicator on each goal, the computer 1008 may estimate fulfilled percentage of each goal. Progress indicators are goal specific and are defined when defining the specific goals for the patient. For example, if the goal is to reduce social anxiety, progress indicators might be increased social interactions and reduced avoidance of social situations as well as lower self-reported anxiety levels in social settings. Based on averaged progress toward therapy goals, the computer 1008 may estimate total therapy completion. Although this would be an estimate, not an exact figure, clear trends may be visible.

Regarding the question, “Are the patient's needs met?” The computer 1008 may analyze the patient-therapist interaction using, for example, an encoder network, a transformer network, latent semantic analysis, semantic space theory, or other natural language processing or large language model techniques. One purpose of analyzing the interaction is to generate answers to questions about physiological needs, safety needs, feelings of love and belonging, feelings of esteem, actions toward self-actualization, expressions of autonomy, expressions of relatedness, and actions demonstrating emotional competence. The computer then may categorize answers into 9 grades of dis/agreement to produce a score for each question between [−1, 1]. Another purpose of analyzing the interaction is to identify missing information, e.g., clinically appropriate discussions that have not occurred between the patient and therapist.

Regarding the question, “Are patient's resources utilized during therapy?” The computer 1008 may analyze the patient-therapist interaction using, for example, an encoder network, a transformer network, latent semantic analysis, semantic space theory, or other natural language processing or large language model techniques. One purpose of analyzing the interaction is to identify incidents of communication that indicate engagement of at least the following resource categories: completeness, diversity, sufficiency for therapy, activation, frequency, integration into therapy strategy, impact on patients progress, and activation of identified resources. Categories of resources include essential resources such as emotional, cognitive, social, physical, financial, spiritual, environmental, and educational/vocational resources. The completeness, diversity, sufficiency for therapy, activation, activation frequency of these resources are evaluated as well as the therapist role in resource reinforcement, the integration of resources into therapy strategy and the impact of patient's engagement and progress of the resource. The analysis is performed by an LLM based on the full history of the therapy conversation.

Regarding the question, “To what extent do the therapist and patient develop a functional relationship?” The computer 1008 may analyze the patient-therapist interaction using, for example, an encoder network, a transformer network, latent semantic analysis, semantic space theory, or other natural language processing or large language model techniques. One purpose of analyzing the interaction is to identify incidents of communication that indicate performance by the therapist and/or patient. The therapeutic relationship is evaluated in categories such as effectiveness, goal alignment, emotional attunement, communication clarity, cultural sensitivity, personalization, progress reflection, supportiveness, crisis management, therapeutic alliance, empathy, validation, approach alignment and flexibility. Each category is briefly analyzed by an LLM summarizing findings, key supporting evidence, notable patterns or trends, and actionable insights. A specific question for each category is evaluated on a nine-graded scale of agreement and converted into a numerical performance metric.

Thus, the system 100 can implement a “learning loop” in which a therapist 104 (optionally, a digital therapist as described in commonly owned patent application with attorney docket 07656-P0037A) interacts with a patient 102 (optionally, a digital patient as described in commonly owned patent application with attorney docket 07656-P0038A), and the system 100 intermediates or monitors the interaction and evaluates the interaction using developed metrics (as described in commonly owned patent application with attorney docket 07656-P0039A). The interaction between therapist and patient generates valuable training data, principally for use by other therapists but also useful for the therapist 104. Where the therapist and/or patient are digital, the system 100 provides for a self-learning mechanism with continuous feedback loop. Thus, the system 100 captures and logs interactions between therapist and patient, evaluates the captured data using the developed metrics, and then fine-tunes the digital therapist and/or patient based on the evaluation. The computer continues to evaluate the fine-tuned model, creating a continuous improvement loop, which contributes to the development of increasingly sophisticated digital models for therapy. While the core identity of the digital patient remains stable in the loop, the system updates its character traits through changes in the patient's inner voice and memory of therapy experiences, reflecting growth and progress in response to communications from the therapist.

The system 100 also may rank therapists by diagnosis, that is compiling ratings of therapists' effectiveness categorized by diagnoses of their patients. The system 100 also may provide higher ranked or higher rated therapist responses 120 as feedback to the therapist or therapist reply system 104.

The system 100 may generate multiple potentially useful digital patients and digital therapists and may train each digital therapist against more than one of the digital patients. This approach may alleviate the potential problem of a digital therapist overfitting to a single digital patient. The system 100 may repeatedly reset digital patients to their starting conditions so that the digital therapists may be trained in multiple iterations. The system 100 may track efficacy of various interventions by a digital therapist against various iterations of a digital patient. The system 100 may update one or more digital therapists' professional knowledge 128 based on higher rated therapist responses 120 for a given digital patient.

The present teachings have been described in language more or less specific as to structural, mechanical, and functional features. It is to be understood, however, that the present teachings are not limited to the specific features shown and described, since the apparatus, system, and/or method herein disclosed comprises preferred forms of putting the present teachings into effect.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The use of “first”, “second,” etc. for different features/components of the present disclosure are only intended to distinguish the features/components from other similar features/components and not to impart any order or hierarchy to the features/components, unless explicitly stated otherwise. The phrase “at least one of,” when used with a list of items, means that different combinations of one or more of the listed items may be used, and only one item in the list may be needed. For example, “at least one of: A, B, and C” includes any of the following combinations: A; B; C; A and B; A and C; B and C; and A and B and C.

Other than in the operating examples, or where otherwise indicated, all numbers expressing quantities of ingredients or reaction conditions used herein are to be understood as modified in all instances by the term “about”.

While the present teachings have been described above in terms of specific embodiments, it is to be understood that they are not limited to those disclosed embodiments. Many modifications and other embodiments will come to mind to those skilled in the art to which this pertains, and which are intended to be and are covered by both this disclosure and the appended claims. For example, in some instances, one or more features disclosed in connection with one embodiment can be used alone or in combination with one or more features of one or more other embodiments. It is intended that the scope of the present teachings should be determined by proper interpretation and construction of any claims and their legal equivalents, as understood by those of skill in the art relying upon the disclosure in this specification and the attached drawings.

What is claimed is:

1. A cognitive behavioral therapy training system comprising:
a plurality of digital patients that each have:
a character trait database having a plurality of human character traits;
a message database having a plurality of patient memories;
a computer with access to the character trait database and the message database, said computer having a communication link between said computer and a digital therapist;
software executing on the computer for retrieving a patient memory and a set of character traits for a test patient and formulating an inner voice of the test patient based on the patient memory and the character traits;
software executing on the computer for combining a new message with the inner voice to compose a reply of the test patient;
software executing on the computer to update the patient memory based on the new message and the reply; and
transmitting the reply to the digital therapist using the communication link;
a plurality of digital therapists that each have:
the message database accessible by the computer;
software executing in the computer with access to the message database as well as environmental factors for generating an assessment;
a database of curated messages processed to generate a numerical representation of each curated message for storage together with the curated message;
software executing in the computer for numerically representing the assessment and/or the reply and for searching the curated message database for matches;
software executing in the computer for generating a message using at least one of the reply, the summary, the assessment, and any matching curated content;
software executing in the computer for skeptically analyzing the message and either returning it to the message generating software for revision or forwarding it to the communication channel for transmission to the mobile computing device; and
a training subsystem comprising:
a computer in communication with the plurality of digital patients and the plurality of digital therapists, said computer having access to the message database;
software executing on the computer for analyzing a message history for a particular digital patient to determine improvement or regression over time and/or over volume of communications through the digital patient and the digital therapist; and
software executing on the computer for ranking digital therapists by diagnosis and providing samples of higher ranked replies,
wherein the system generates multiple potentially useful digital patients and digital therapists and trains each digital therapist against more than one of the digital patients.

2. The system of claim 1, wherein the system repeatedly resets digital patients to their starting conditions so that the digital therapists may be trained in multiple iterations.

3. The system of claim 1, wherein the system tracks efficacy of various interventions by a digital therapist against various iterations of a digital patient.

4. The system of claim 1, wherein the system updates one or more digital therapists' professional knowledge based on higher rated therapist responses for a given digital patient.

5. The system of claim 1, wherein the software for ranking therapists implements a semantic analysis technique on a plurality of sequences of messages and replies, each of the sequences being associated with a patient and a therapist.

6. The system of claim 5, wherein the software ranks a plurality of therapists based on respective assessments of state, development, and/or progress in therapy for one or more patients associated with each of the plurality of therapists.

7. The system of claim 1, wherein the software for ranking therapists analyzes the message history using a semantic technique to assess each reply against key attributes of therapeutic praxis, said attributes including at least two of: engaging, helpful, language, empathic, actionable, relevant, accurate, appropriate, accepting, clear, empowering.

8. The system of claim 1, wherein the software for ranking therapists analyzes the message history using a semantic technique to assess at least one of the patient's emotional spectrum, development, and activation.

9. The system of claim 1, wherein the software for ranking therapists provides higher ranked or higher rated therapist responses as feedback to the therapist reply system.

10. The system of claim 1, wherein the software executing on the computer is adapted to generate synthetic training data for the patient messaging system and/or the therapist reply system.
